(19) United States
(12) Patent Application Publication
(10) Pub. No.: US 2004/0097486 A1
(43) Pub. Date: May 20, 2004

(54) Use of an H1 antagonist and a safe steroid to treat eye conditions

(76) Inventor: John M. Yanni, Burleson, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/704,254

(22) Filed: Nov. 7, 2003

Related U.S. Application Data
(63) Continuation-in-part of application No. 10/069,851, filed on Feb. 28, 2002, now Pat. No. 6,649,602.
Application No. 10/069,851 is a 371 of international application No. PCT/US00/29436, filed on Oct. 26, 2000.
(60) Provisional application No. 60/166,194, filed on Nov. 18, 1999.

(51) Int. Cl. A61K 31/55; A61K 31/496; A61K 31/495; A61K 31/473; A61K 31/454

(52) U.S. Cl. 514/217.05; 514/255.04; 514/290; 514/254.02; 514/326

(57) ABSTRACT

Compositions and methods for treating VKC, GPC, AKC and allergic conjunctivitis with H₁ antagonists and ocularly safe steroids are disclosed.

This application is a continuation-in-part of U.S. application Ser. No. 10/069,851, filed Feb. 28, 2002, which claims priority from PCT Application No. U.S. 60/29436, filed Oct. 26, 2000, which claims priority from U.S. No. 60/166,194, filed Nov. 18, 1999.

The present invention is directed to the use of an H₁ antagonist in combination with an ocularly safe steroid to treat ocular conditions, specifically vernal keratoconjunctivitis (VKC), giant papillary conjunctivitis (GPC), atopic keratoconjunctivitis (AKC), and allergic conjunctivitis.

### BACKGROUND OF THE INVENTION

Vernal keratoconjunctivitis (VKC), giant papillary conjunctivitis (GPC), and atopic keratoconjunctivitis (AKC) have historically been treated with a regimen of oral or topical antihistamines and/or oral or topical steroids with varying degrees of success (when used individually). Systemic treatment typically requires higher concentrations of the drug compound to be administered to afford an effective concentration to reach the necessary treatment site. Antihistamine compounds are known to have central nervous system (CNS) activity, which manifests itself in drowsiness and may have anticholinergic activity which manifests itself in the drying of mucus membranes. Steroid therapy also has significant systemic side effects, including the elevation of intraocular pressure (IOP). Topical ocular use of steroids can also cause a rise in IOP and induce cataract formation.

Topical ocular combination therapy is known. For example, U.S. Pat. No. 5,192,780 (York, et al) discloses the use of an antihistamine and an antiallergic for treating ophthalmic allergic responses. U.S. Pat. No. 5,149,694 (Cagle, et al.) discloses compositions of tobramycin and dexamethasone for the control of infection and inflammatory response.

The use of an H₁ antagonist in combination with a safe steroid for treating VKC, GPC, AKC and allergic conjunctivitis is not known.

### SUMMARY OF THE INVENTION

The present invention is directed to compositions of combinations of H₁ antagonists and safe steroids to treat VKC, GPC, AKC and allergic conjunctivitis. Methods for the use of the compositions in mammals are also contemplated.

### DESCRIPTION OF PREFERRED EMBODIMENTS

The current invention comprises compositions of H₁ antagonists for treating the itching, redness, and edema associated with VKC, GPC, AKC and allergic conjunctivitis. The compositions also include a safe steroid, as used herein the term “safe steroid” means a steroid which treats eosinophil and neurotrophil associated inflammation, reduces papillae formation, and which is effective in treating inflammation without causing a clinically significant elevation in IOP.

The H₁ antagonists which are useful according to the present invention include all efficacious compounds, including, but not limited to: emedastine, levocabastine, mequitazine, chlorpheniramine, brompheniramine, astemizole, cetirizine, terfenadine, rocastine, loratadine, 5-[2-[4-bis(4-fluorophenyl)hydroxymethyl-1-piperidinyl]ethyl]-3-methyl]-2-oxazolidinone ethanedioate, pyrilamine, clemastine, azelastine, ketotifen, olopatadine, epinastine and mapinastine.

Safe steroids which can be used herein include any glucocorticoid which meets the safe steroid definition, including but not limited to, fluoromethalone, rimexolone, loteprednol, dexamethasone beloxil and its analogues described in U.S. Pat. Nos. 5,223,493 and 5,420,120.

The H₁ antagonists and safe steroids (compounds) can be incorporated into various types of ophthalmic formulations for delivery to the eye. These compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving the compound in a physiologically acceptable isotonic aqueous buffer. Further, the ophthalmic solution may include an ophthalmologically acceptable surfactant to assist in dissolving the compound. The ophthalmic solution may also contain a thickener such as hydroxymethylcellulose, hydroxyethylcellulose, hydroxypropylmethylcellulose, methylcellulose, polyvinylpyrrolidone, or the like, to improve the retention of the formulation in the conjunctival sac. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated.

The compounds are preferably formulated as topical ophthalmic suspensions or solutions, with a pH of about 6.0 to 8.0. The H₁ antagonists will normally be contained in these formulations in an amount 0.01% to 0.3% by weight, but preferably in an amount of 0.05% to 0.1% by weight. The safe steroids will normally be contained in those formulations in an amount 0.05% to 1.5% by weight, preferably in an amount of 0.05% to 1.0% by weight, and most preferably in an amount of 0.05% to 0.5% by weight. Thus, for topical presentation 1 to 2 drops of these formulations would be delivered to the surface of the eye up to 4 times per day according to the routine discretion of a skilled clinician.

In one embodiment where the H₁ antagonist is emedastine, the preferred compositions of the present invention include 0.01% to 0.05% emedastine and 0.1% to 1.0% dexamethasone beloxil or loteprednol. In another embodiment, where the H1 antagonist is olopatadine, the preferred compositions of the present invention include 0.1% olopatadine and 0.075% rimexolone.

The following example is illustrative of the composition of the present invention, but in no way limiting.

### EXAMPLE 1

| Ingredient | Weight % |
|---|---|
| Emedastine | 0.05 |
| Dexamethasone beloxil | 0.1 |
| Hydroxypropyl methylcellulose | 0.5 |
| Dibasic sodium phosphate | 0.2 |
| Disodium EDTA | 0.01 |
| Sodium Chloride | 0.75 |
| Polysorbate 80 | 0.01 |
| Benzalkonium chloride | 0.01 |
| Sodium hydroxide, hydrochloric acid | adjust to approx. 7.0 |
| Water | q.s. 100 |

### EXAMPLE 2

| Ingredient | Weight % |
|---|---|
| Olopatadine HCl | 0.111* |
| Rimexolone | 0.075 |
| Benzalkonium Chloride | 0.01 |
| Povidone K90 | 1.5 |
| Dibasic sodium phosphate | 0.2 |
| Disodium EDTA | 0.01 |
| Sodium Chloride | 0.5-0.8 |
| Polysorbate 80 | 0.01 |
| Sodium hydroxide, hydrochloric acid | adjust to approx. 7.0 |
| Water | q.s. 100% |

*0.111% olopatadine HCl is equivalent to 0.1% olopatadine

### EXAMPLE 3

| Ingredient | Weight % |
|---|---|
| Olopatadine HCl | 0.111* |
| Rimexolone | 0.075 |
| Polyquaternium-1 | 0.0005-0.001 |
| Povidone K90 | 1.5 |
| Dibasic sodium phosphate | 0.2 |
| Disodium EDTA | 0.01 |
| Sodium Chloride | 0.5-0.8 |
| Polysorbate 80 | 0.01 |
| Sodium hydroxide, hydrochloric acid | adjust to approx. 7.0 |
| Water | q.s. 100% |

*0.111% olopatadine HCl is equivalent to 0.1% olopatadine

We claim:

1. A method of treating ocular conditions in mammals selected from the group consisting of vernal keratoconjunctivitis, giant papillary conjunctivitis, atopic keratoconjunctivitis, and allergic conjunctivitis which comprises administering a pharmaceutically effective amount of a composition comprising an H1 antagonist and a safe steroid, wherein the amount of H1 antagonist in the composition is 0.01-0.3% by weight and the amount of safe steroid in the composition is 0.05-1.5% by weight.

2. The method of claim 1 wherein the H1 antagonist is selected from the group consisting of emedastine; levocabastine; mequitazine; chlorpheniramine; brompheniramine; astemizole; cetirizine; terfenadine; rocastine; loratadine; 5-[2-[4-bis(4-fluorophenyl)hydroxymethyl-1-piperidinyl] ethyl]-3-methyl]-2-oxazolidinone ethanedioate, pyrilamine; clemastine; azelastine; ketotifen; olopatadine; epinastine; and mapinastine.

3. A method of treating ocular conditions in mammals selected from the group consisting of vernal keratoconjunctivitis, giant papillary conjunctivitis, atopic keratoconjunctivitis, and allergic conjunctivitis which comprises administering a pharmaceutically effective amount of a composition comprising an H1 antagonist and a safe steroid, wherein the H1 antagonist is olopatadine and the safe steroid is rimexolone.

4. The method of claim 3 wherein the amount of H1 antagonist in the composition is 0.01-0.3% by weight.

5. The method of claim 4 wherein the amount of safe steroid in the composition is 0.05-1.5% by weight.

6. The method of claim 5 wherein the amount of safe steroid in the composition is 0.05-1.0% by weight.

7. The method of claim 6 wherein the amount of safe steroid in the composition is 0.05-0.5% by weight.

8. A method of treating ocular conditions in mammals selected from the group consisting of vernal keratoconjunctivitis, giant papillary conjunctivitis, atopic keratoconjunctivitis, and allergic conjunctivitis which comprises administering a pharmaceutically effective amount of a composition comprising 01-0.3% by weight of olopatadine and 0.05-0.5% by weight of rimexolone.
